(19) United States
(12) Patent Application Publication
(10) Pub. No.: US 2022/0409130 A1
(43) Pub. Date: Dec. 29, 2022

(54) WEARABLE STETHOSCOPE AND ITS RELATED MONITORING SYSTEM

(71) Applicant: Decentralized Biotechnology Intelligence Co., Ltd., New Taipei City (TW)

(72) Inventors: Yao-Sheng Chou, Taipei City (TW); YEN-HAN CHOU, New (TW)

(21) Appl. No.: 17/516,667

(22) Filed: Nov. 1, 2021

(30) Foreign Application Priority Data
Jun. 23, 2021 (TW) 110122985

(51) Int. Cl. A61B 5/00; H04R 1/46; H04R 19/04; H04R 3/04; A61B 7/04; A61B 5/318; A61B 5/282; A61B 5/308; A61B 5/16

(52) U.S. Cl. CPC A61B 5/6801; H04R 1/46; H04R 19/04; H04R 3/04; A61B 7/04; A61B 5/318; A61B 5/7203; A61B 5/282; A61B 5/308; A61B 5/0006; A61B 5/0022; A61B 5/16; H04W 88/02

(57) ABSTRACT

A wearable stethoscope includes a sound sensing device for collecting heart sound signals of the body, an electrocardiogram sensing device for collecting electrocardiogram signals of the body, a processing unit, powered by a power source, coupled to the sound sensing device and the electrocardiogram sensing device to perform data preprocessing on the above-mentioned signals to remove background noise. An external electronic computing device is set up to analyze and process the fed pre-processed ECG signal and heart sound signal, perform feature extraction in combination with the user's physiological parameters and medical records to obtain related feature vectors, input the feature vectors into a screening model, obtain an evaluation value and give corresponding suggestions. After screening, users can upload the verification results to the cloud database to expand the existing training samples for further optimizing the parameters of the screening model.

### CROSS-REFERENCE TO RELATED APPLICATIONS

This patent application is based on, and claim priority from TAIWAN patent application serial number 110122985, filed on Jun. 23, 2021, the disclosure of which is hereby incorporated by reference herein in its' entirety.

### TECHNICAL FIELD

The present invention relates to a device and system for monitoring the physiological activities of the human body through a mobile device, and more particularly, a wearable stethoscope device and a corresponding monitoring system.

### BACKGROUND OF RELATED ARTS

A stethoscope is an instrument for diagnosing the activity of organs by detecting their sounds. The electronic stethoscope collects the sounds of heart, lungs and other organs by placing the earpiece on the corresponding part of the tested organism, and converts these sounds into electrical signals. After amplification, these sounds are directly emitted by the speaker and can be collected, so that doctor or related medical personnel can justify the cause or lesion of the disease and make a correct diagnosis based on the corresponding collected sound signals.

Heart sounds are noises generated by the beating heart and the resultant flow of blood through it, which can be transmitted to the chest wall through the surrounding tissues. Specially, the sounds reflect the turbulence created when the heart valves snap shut. In cardiac auscultation, an examiner may use a stethoscope to listen for these unique and distinct sounds that provide important auditory data regarding the condition of the heart. Certain abnormal heart activities can produce murmurs or other abnormal heart sounds. Therefore, listening to heart sounds or recording a phonocardiogram (PCG) can effectively make up for the deficiency of cardiac auscultation.

Heart failure is a prevalent public health problem worldwide and poses a huge burden on overall medical costs. In recent years, people has increasingly concerned about their health issues, methods of health management by monitoring people's physical and mental states in daily life have begun to spread, which are performed by recording and analyzing physiological information over a long period of time (from several hours to several months).

In terms of obtaining people's physiological information, it includes heartbeat rate or R-R interval, electrocardiogram (ECG) waveform, step counts, physical activity level, body acceleration, etc. By monitoring these physiological information in daily life, it can be effectively used for health improvement, or for early detection of diseases, etc. Based on the above motivations, the present invention is intended to make improvements on traditional stethoscope and its related monitoring system.

### SUMMARY

Microelectromechanical systems (MEMS) can be applied to wearable devices. MEMS sensor has advantages including its small size, low power consumption, low cost, and integration and embedding capabilities with hardware. Beneficial to the advancement of MEMS device manufacturing technology, various sensors based on MEMS technology can be easily embedded in handheld and wearable devices, making technology applications ubiquitous.

Compared with traditional auscultation devices, embedded electronic auscultation devices integrated MEMS sensors with hardware, which have characteristics of small size, low power consumption, and low cost, can provide miniaturized and wearable functions. Combining the characteristics of the internet of things (IoT) and cloud analysis, embedded electronic auscultation devices can be used for long-term recording and analysis of persons' physiological information, monitoring their physical and mental state in daily life, or detecting their vital signs to predict possible urgent and critical health risks.

Based on the above description, the present invention proposes a wearable body sound signal capturing device, such as a wearable stethoscope and a corresponding monitoring system, to solve the deficiencies existed in the prior art.

A wearable stethoscope includes a sound sensing device for collecting heart sound signals of a body, an electrocardiogram sensing device for collecting electrocardiogram signals of the body, a processing unit, powered by a power source, coupled to the sound sensing device and the electrocardiogram sensing device to perform data preprocessing on the above-mentioned signals to remove background noise, and the sound sensing device is sound sensor with an embedded microelectromechanical system (MEMS).

In one embodiment, the aforementioned MEMS sound sensor is a capacitive sound sensing device based on the MEMS technology.

In one embodiment, the above-mentioned capacitive sound sensing device detects sound pressure through measuring its nominal capacitance changes.

In one embodiment, the processing unit includes a filter electrically connected to the sound sensing device and the electrocardiogram sensing device for receiving the body sound signals and the electrocardiogram signals and filtering them, a signal amplifier electrically connected to the filter for amplifying both the body sound signals and electrocardiogram signals been filtered, an analog-to-digital converter electrically connected to the filter for converting both the body sound signals and electrocardiogram signals that have been filtered and amplified into digitalized body sound signals and electrocardiogram signals, a microprocessor electrically connected to the analog-to-digital converter for receiving the digitalized body sound signals and electrocardiogram signals and performing processing operation to obtain pre-processed body sound signals and electrocardiogram signals, both signals been de-noising by the microprocessor, wherein the microprocessor executes instructions to store the pre-processed body sound signals and electrocardiogram signals in a storage device electrically connected to the microprocessor, or the pre-processed body sound signals and electrocardiogram signals are sent to an external mobile device for further analysis through a wireless transmission device electrically connected to the microprocessor.

A monitoring system of a wearable stethoscope includes a wearable stethoscope describe above, an external electronic computing device communicatively coupled to the wearable stethoscope for acquiring the pre-processed electrocardiogram signal and body sound signal, and a cloud database communicatively coupled to the external electronic computing device, wherein the processing unit of the external electronic computing device is configured to execute instructions stored in the storage unit of the external electronic computing device, the instructions include analyzing and processing the fed pre-processed electrocardiogram signals and body sound signals, performing feature extraction in combination with the user's physiological parameters and medical records to obtain related feature vectors, inputting the feature vector into a screening model, obtaining an evaluation value and giving corresponding suggestions. After screening, users can upload verification results to the cloud database to expand the existing training samples for further optimizing the parameters of the screening model.

In one embodiment, verification results includes the related feature vectors obtained from the feature extraction, evaluation value and the corresponding suggestions

In one embodiment, the external electronic computing device is a smart phone.

In one embodiment, the external electronic computing device is a cloud server.

In one embodiment, the screening model is an algorithm constructed based on comparing the relevant physiological parameters between patients and healthy persons.

In one preferred embodiment, the algorithm is a computing program or an application program executable by the external electronic computing device.

### BRIEF DESCRIPTION OF THE DRAWINGS

The components, characteristics and advantages of the present invention may be understood by the detailed descriptions of the preferred embodiments outlined in the specification and the drawings attached:

FIG. 1 illustrates the proposed system architecture according to a preferred embodiment of the present invention.

FIG. 2 illustrates a functional block diagram of the wearable stethoscope according to a preferred embodiment of the present invention.

FIG. 3 illustrates an example of a processing system of a mobile device or a computing system operating on a cloud server according to a preferred embodiment of the present invention.

FIG. 4 illustrates a flow chart of psychogenic diseases screening according to a preferred embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

The present invention provides a wearable body sound signal capturing device combined with MEMS sensors, such as a wearable stethoscope, which combines an embedded MEMS sensing system and wireless transmission system (for example, Bluetooth or Wi-Fi wireless transmission system), as a portable body acoustic signal collecting device, which can be connected to the internet of things (IoT). Collected personal heart sound signals, lung sound signals, abdominal sound signals, fetal sound signals, and ECG information or other physiological data can be transmitted and stored in a cloud server.

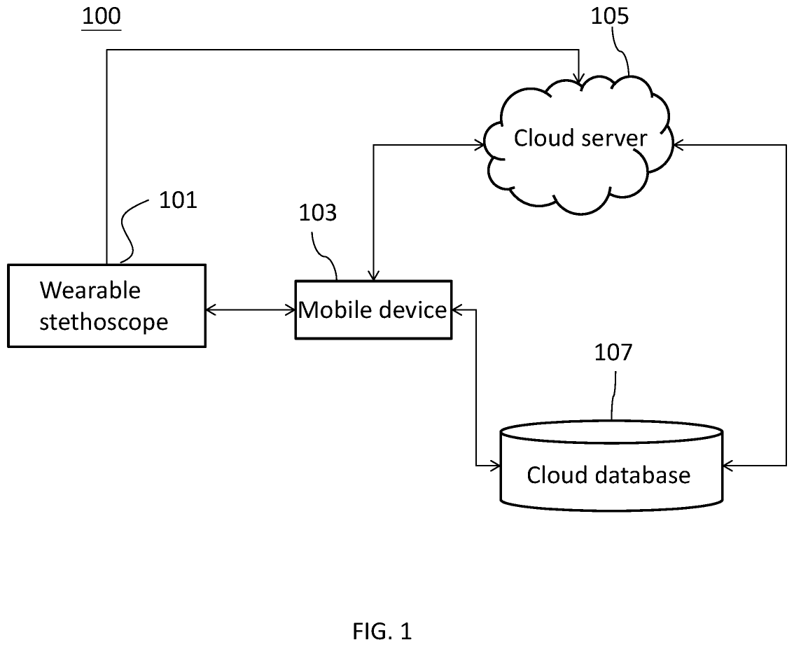

FIG. 1 illustrates the proposed system architecture 100 mentioned above, which includes a wearable body sound signal capturing device communicatively coupled to a mobile device 103, such as a smart phone, for collecting heart sound signals of the body, the data collected by the wearable stethoscope (i.e., wearable body acoustic signal capturing device) 101, such as personal heart sound signals, lung sound signals, abdominal sound signals, fetal sound signals, ECG information, or other physiological data, can be received by the mobile device 103 and uploads to the cloud server 105 via the network by wireless transmission function of the mobile device 103, and the data will be stored in the cloud database 107. Alternatively, the mobile device 103 can be replaced by tablet, notebook computer, or similar electronic computing devices, or wearable electronic devices, such as smart watch, bracelet, glasses etc. for providing wireless internet accesses. The system also includes an application program installed on the mobile device 103, which includes instructions for receiving and sending data between the wearable stethoscope 101, the mobile device 103, and the cloud server 105. The application programs can be operated based on Android, Windows 10 or iOS operating system, it can also upload the collected data to the cloud server 105 for storage and/or processing.

The above system architecture 100 can continuously monitor the electrical and mechanical activities of the heart through the mobile device 103, store collected data in the cloud server 105, and execute programs on the mobile device 103 or/the cloud server 105, for example, execute algorithms for detecting cardiac abnormalities and extracting features of ECG signals.

Alternatively, the proposed system architecture 100 includes a wearable body sound signal capturing device can communicatively coupled to the cloud server 105 directly via wireless transmission such as WiFi, 4G/5G, etc. without need a mobile device 103, for collecting heart sound signals of the body, the data collected by the wearable stethoscope (i.e., wearable body acoustic signal capturing device) 101 can be uploaded to the cloud server 105. The cloud server 105 also includes an application program installed, which includes instructions for receiving and sending data between the wearable stethoscope 101 and the cloud server 105. The application programs can be operated based on Android, Windows 10 or iOS operating system, it can also upload the collected data to the cloud server 105 for storage and/or processing.

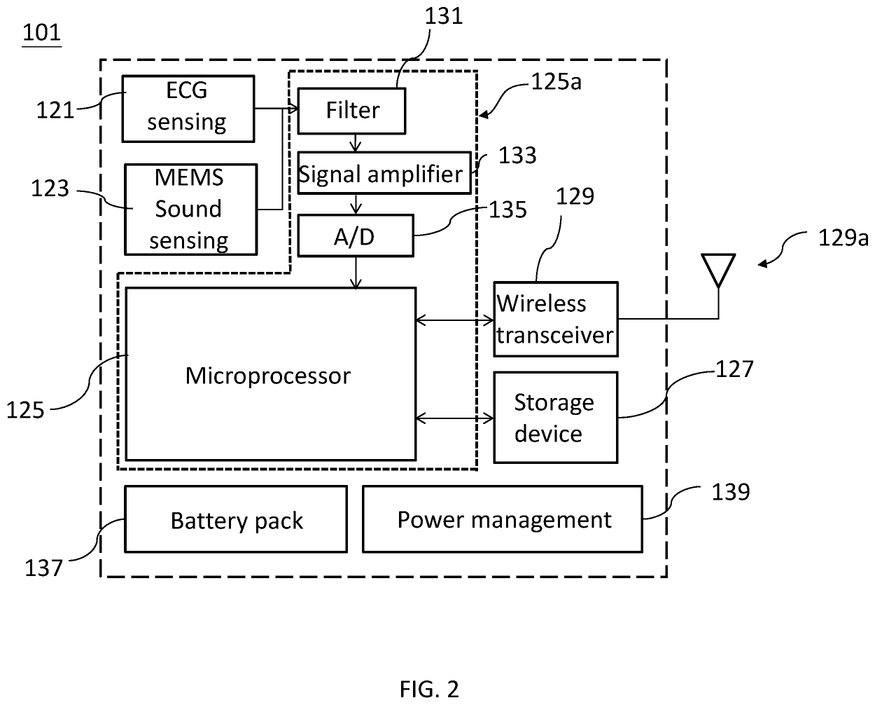

The aforementioned wearable body sound signal capturing device 101 is a wearable stethoscope with an embedded MEMS system or a mini microphone, which is manufactured on a flexible circuit board, it can also be integrated into clothing (for example, functional sports clothing), and its functional block diagram is shown in FIG. 2. The wearable body sound signal capturing device 101 can respectively obtain ECG signals and body sound signals from the human body through at least one (but not limit to one) electrocardiogram (ECG) sensing device 121 and MEMS sound sensing device 123, and can be configured to monitor physiological data for remote diagnosis. In one embodiment, the MEMS sound sensing device 123 is a capacitive sound sensing device based on MEMS technology to detect sound pressure (body sound signal) through measuring its nominal capacitance changes. In one embodiment, the above-mentioned ECG sensing device uses a three-lead electrocardiogram wire with three electrodes (positive, negative, and ground) to detect electrocardiographic (ECG) signals. The wearable body sound capturing device 101, which includes a microprocessor, a storage unit, and a wireless transmission unit, can receive and send data, and execute software applications. The aforementioned ECG sensing devices 121 and MEMS sound sensing devices 123 can be separately or simultaneously implanted into the wearable stethoscope (wearable body sound signal capturing device) 101 of the present invention.

The microprocessor 125 can be a microcontroller, a digital signal processor (DSP), an application-specific integrated circuit (ASIC), a programmable logic circuit, or other digital data processing devices that can execute instructions to perform operations. The microprocessor 125 can execute various application programs stored in the storage device 127.

The storage device 127 may include read-only memory (ROM), random access memory (RANI), electrically erasable programmable ROM (EEPROM), flash memory, or any other kind memories that are regularly used in computers.

The wireless transceiver 129 is connected to an antenna 129a, and the antenna 129a is configured to send output data and receive input data through a wireless communication channel. The wireless telecommunication channel can be a digital wireless telecommunication channel, such as WiFi, Bluetooth, RFID, NFC, 3G/4G/5G or any other future developed wireless communication interface.

The above-mentioned ECG signals and body sound signals obtained from the human body through a plurality of ECG electrodes 121 and MEMS sound sensing devices 123 are filtered by the filter 131 and amplified by the signal amplifier 133. ECG and body sound signals been filtered and amplified are passed through an analog-to-digital converter (ADC) 135, which converts the analog signal into a digital signal, and then are processed by the microprocessor 125 to obtain ECG and body sound signals that are de-noising and stable. The microprocessor 125 can store the ECG and body sound signals that are de-noising and stable in the storage unit through instructions or programs, or send these signals to the mobile device, such as smart phone, via its wireless transmission unit 129 for performing further data analysis.

The battery pack 137 provides power for the wearable body sound signal capturing device 101, and can be coordinated with the power management device 139 to optimize overall power usage.

The aforementioned signal amplifier 133, filter 131, analog-to-digital converter 135, and microprocessor 125 can be integrated into an integrated circuit (IC) as a processing unit 125a of the wearable stethoscope 101.

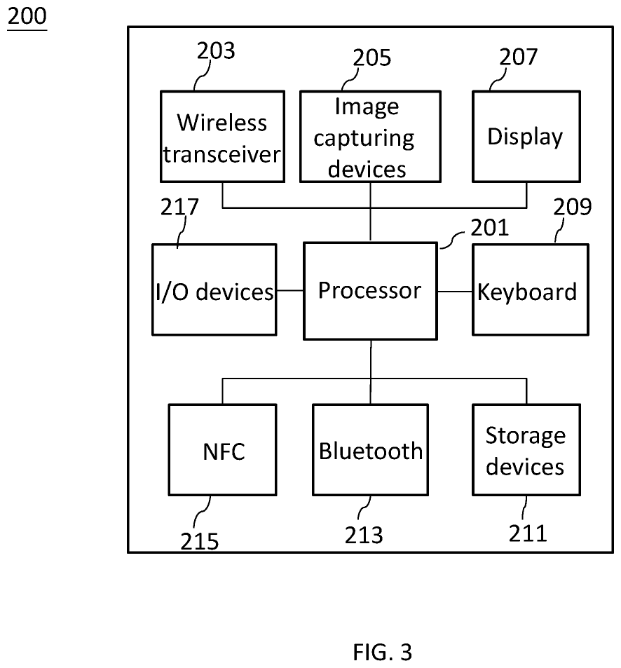

FIG. 3 illustrates an example of a processing system 200 of a mobile device 103 or a computing system operating on a cloud server 105. Specifically, the processing system 200 represents a processing system of the mobile device 101 or a computing system that operates on a cloud server 105 (FIG. 1). The cloud server 105 (or a mobile device) executes commands to perform the operational processes according to embodiments the present invention, such as the algorithm for detecting cardiac abnormalities, and extracting of ECG signal features as described previously. A person skilled in the art should understand that, without departing from the spirit of the present invention, the above-mentioned commands can be stored and/or executed as hardware, software or firmware. In addition, the exact configuration of each processing system may be different, and the processing system 200 shown in FIG. 3 is only an example.

The processing system 200 includes a processor 201, a wireless transceiver device 203, image capture devices 205, a display 207, a keyboard area 209, storage devices 211, a Bluetooth device 213, a near field communication (NFC) device 215, and I/O devices 217.

Wireless transceiver device 203, image capturing devices 205, display 207, keyboard area 209, storage devices 211, Bluetooth device 213, near field communication (NFC) device 215, I/O devices 217, and any number of other peripheral devices are electrically connected to the processor 201 to exchange data with the processor 201 and to use these data in applications executed by the processor 201.

The wireless transceiver 203 is connected to an antenna, and is configured to transmit and output sound and data signals and receive sound and data signals through a wireless telecommunication channel. In one embodiment, the wireless telecommunication channel can be a digital wireless telecommunication channel, such as WiFi, Bluetooth, RFID, NFC, 3G/4G/5G or any other future developed wireless communication interface.

The image capturing devices 205 can be any device capable of capturing still and/or moving images, such as a complementary metal oxide semiconductor (CMOS) or a charge coupled sensor type cameras. The display 207 receives data that can be displayed from the processor 201 and displays images on the screen for the user to watch. The display 207 can be a liquid crystal display (LCD) or an organic light emitting diode (OLED) display. The keyboard area 209 receives user's input information and sends the input to the processor 201. In one embodiment, the display 207 can be acted as a touch sensitive surface and used as the keyboard area 209 to receive user's input information.

The storage devices 211 can be devices that send data to and receive data from the processor 201 and store the received data. The storage devices 211 may include non-volatile memories, such as a read-only memories (ROMs), which store required instructions and data to operate individual subsystems of the processing system and guide the system at startup stage. A person skilled in the art should understand that any number of memories can be used to perform this function. The storage devices 211 may also include a volatile memories, such as random access memories (RAMs), which store data or programs executed by the processor 201 that required to perform the operational processes according to embodiments the present invention. A person skilled in the art should understand that any type of memory can be used as a volatile memory, and the exact type used is left to those skilled in the art as a design choice.

The Bluetooth device 213 is a device that allows the processing system 200 to establish communication with similar devices such as the wearable stethoscope 101 based on the Bluetooth technology standard. The near field communication (NFC) device is a device that allows the wearable auscultation device 101 and another similar device to establish wireless communication by being touch together or being in close proximity to each other.

Other peripheral devices that can be connected to the processor 201 include a global positioning system (GPS) and other positioning transceivers.

The processor 201 can be a processor, a microprocessor, or any combination of a processor and a microprocessor, which executes and processes the operation instructions according to the present invention. The processor 201 can execute various application programs stored in the storage devices. These applications can receive user's input information via the display 207 with a touch screen or directly via the keyboard area 209. Some application programs stored in the storage devices 211 that can be executed by the processor 201 may be application programs developed by UNIX, Android, iOS, Windows, Blackberry, or other operating systems.

Body sound signals, such as heart sounds, can effectively reflect the heart situation, especially valve activity and blood flow. For example, the first heart sound is mainly produced by the closure of the atrioventricular valve, and the second heart sound is mainly produced while the semilunar valve is closed. For many cardiovascular diseases, especially valvular diseases, heart sounds are important diagnostic information, so they are widely used in clinical practice.

Heart sound segmentation is the basis and prerequisite for establishing a screening and decision-making system of psychogenic disease, and its purpose is to locate the main components of heart sounds (first heart sound S1, systolic period, second heart sound S2, and diastolic period), which can provide positioning benchmarks for characteristic extraction and pattern discrimination of heart sound. Success of the decision-making system is highly depended on the accuracy of the heart sound segmentation. The ECG signal can be used as a reference signal for the heart sound segmentation. In timely order, the R wave of the ECG signal matches the heart sound signal S1 and S2.

The wearable body acoustic signal capturing device 101 shown in FIG. 2, which obtains the ECG signal of the heart and the body sound signal (including the heart sound signal) through a plurality of ECG sensing electrodes 121 and a MEMS sound sensing device 123 from the user.

According to one embodiment of the present invention, the ECG signal of the heart and the body sound signal pass through the output ends of the plurality of ECG sensing electrodes 121 and the MEMS sound sensing device 123 are processed by signal amplifiers, filters, and analog-to-digital converters to remove their background noise and digitized these signals (i.e., ECG and body sound signals). These processed signals are stored in the storage device 127 of the wearable auscultation device 101.

In one embodiment, the digitized ECG signal and body sound signal stored in the storage device 127 of the wearable body audio capture device 101 can be sent to the processing system 200 of the mobile device 103 or to a processing system 200 operated on a computing system been acted as a cloud server 105 via wireless transmission for storage and further processing and analysis.

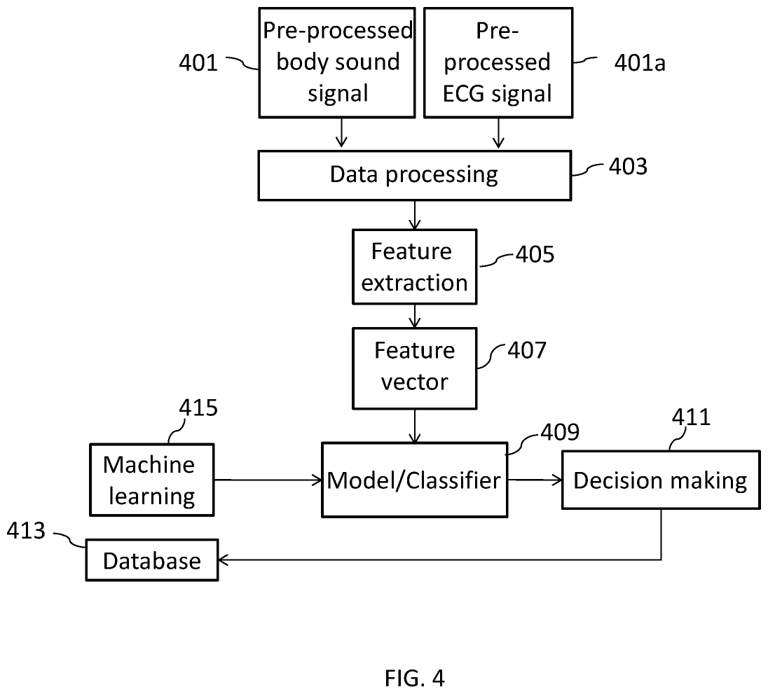

In an embodiment, a flow chart demonstrating the screening process for psychogenic diseases is shown in FIG. 4. The processor of the processing system 200 (refer to FIG. 3), which can be a mobile device, such as a smart phone/or a processor set by an external computing electronic device such as a cloud server, is configured to execute instructions/commands stored in a storage device of an external electronic computing device. The screening process for psychogenic diseases performed by the processor includes performing data analysis and processing on the pre-processed ECG signals 401a and body sound signals 401 that are fed in with their background noise been removed to obtain corresponding body sound data and ECG data (step 403); combining the heart sound data of the body sound signal data and electrocardiogram data together with the user's physiological parameters and medical records for feature extraction (step 405), and obtaining related feature vectors (step 407); inputting the above-mentioned feature vector into a screening model (Model/Classifier, step 409) to obtain an evaluation value (decision making, step 411), and giving corresponding suggestions based on the evaluation value; and uploading the verification results to a remote data database (step 413) by the user after screening, expanding the existing training samples (machine learning, step 415) to further optimize the parameters of the screening model.

In an embodiment, the above-mentioned pre-processed ECG signals 401a and body sound signals 401 also include their corresponding ECG and heart sound signal segmentations after de-noising stage.

In an embodiment, the feature extraction, which is the process of identifying distinct properties from a signal. For ECG signals, wavelet transformation is used to decompose the ECG pulses into their corresponding wavelet coefficients and select a handful of points as their features. For heart sound (HS) signals, the signal features can be extracted from the HS signals from, for example, time domain feature extraction technique to extract timing, intensity, frequency location over time and shape, zero crossing rate, power, instantaneous energy, systole duration, diastole duration, durations of S1 and S2, etc., or from frequency domain feature extraction technique to extract spectral power based features, instantaneous frequency, dominant maximum frequency, etc.

In an embodiment, ECG and HS classification can be performed by the artificial neural network (ANN) and support vector machine (SVM) algorithms.

In an embodiment, the above-mentioned screening model is an algorithm constructed by comparing the relevant physiological parameters between the patient and normal person.

In an embodiment, the above-mentioned verification results includes the related feature vectors obtained from the feature extraction, evaluation value and the corresponding suggestions.

In one embodiment, the above-mentioned algorithm is a computing program or an application program executable by the external electronic computing device.

In an embodiment, the external electronic computing device can be a mobile device (for example, a smart phone) or a cloud server.

In another embodiment, the above-mentioned wearable electrocardiogram device includes an electrocardiographic measuring device for collecting electrocardiographic signals of the driver's body, a processing unit powered by a power source and coupled to the electrocardiographic measuring device for pre-processing collected the ECG signal to remove background noise, and a transmission module, which transmits the received signal to a vehicle's on-board driver recognition system to prevent intrusion or unauthorized usage of the vehicle.

In one embodiment, the above-mentioned power source is coordinated with a power management device to optimize overall power usage.

In one embodiment, the above-mentioned wireless transmission device is a wireless transceiver connected to an antenna and is configured to transmit and receive data through a wireless telecommunication channel.

While various embodiments of the present invention have been described above, it should be understood that they have been presented by a way of example and not limitation. Numerous modifications and variations within the scope of the invention are possible. The present invention should only be defined in accordance with the following claims and their equivalents.

What is claimed is:

1. A wearable stethoscope comprising:
a processing unit powered by a power source;
a sound sensing device coupled to said processing unit to collect body sound signals of a body;
an electrocardiogram sensing device coupled to said processing unit to collect electrocardiogram signals of said body;
wherein said processing unit is employed to perform data preprocessing on said collected heart sound signals and electrocardiogram signals to remove background noise; and
wherein said sound sensing device is a sound sensor with an embedded microelectromechanical system (MEMS).

2. The wearable stethoscope of claim 1, wherein said sound sensor with an embedded MEMS system is a capacitive sound sensing device based on MEMS technology.

3. The wearable stethoscope of claim 2, wherein said capacitive sound sensing device detects sound pressure through measuring nominal capacitance changes.

4. The wearable stethoscope of claim 1, wherein said electrocardiogram sensing device includes three electrodes (positive, negative, and ground) to detect said electrocardiographic signals.

5. The wearable stethoscope of claim 1, wherein said processing unit includes:
a filter electrically connected to said sound sensing device and said electrocardiogram sensing device to receive said body sound signals and said electrocardiogram signals to filter said body sound signals and said electrocardiogram signals;
a signal amplifier electrically connected to said filter to amplify said filtered body sound signals and said filtered electrocardiogram signals;
an analog-to-digital converter electrically connected to said filter to convert said filtered body sound signals and said filtered electrocardiogram signals been amplified into digitalized body sound signals and electrocardiogram signals.

6. The wearable stethoscope of claim 5, further comprising a microprocessor electrically connected to said analog-to-digital converter to obtain pre-processed body sound signals and electrocardiogram signals.

7. The wearable stethoscope of claim 6, wherein said microprocessor executes instructions to store said pre-processed body sound signals and electrocardiogram signals in a storage device electrically connected to said microprocessor.

8. The wearable stethoscope of claim 6, wherein said pre-processed body sound signals and electrocardiogram signals are sent to an external electronic computing device for further analysis through a wireless transmission device electrically connected to said microprocessor, said wireless transmission device is a wireless transceiver connected to an antenna and is configured to transmit and receive data through a wireless telecommunication channel.

9. The wearable stethoscope of claim 1, wherein said wearable stethoscope is integrated into clothing.

10. The wearable stethoscope of claim 6, wherein said microprocessor is a microcontroller, a digital signal processor (DSP), an application-specific integrated circuit (ASIC), a programmable logic circuit, or other digital data processing devices that can execute instructions to perform operations.

11. A monitoring system of a wearable stethoscope, comprising:
a processing unit, powered by a power source, said wearable stethoscope coupled to said processing unit, said wearable stethoscope collecting body sound signals and electrocardiogram signals of a body, wherein said processing unit is employed to perform data preprocessing on said collected heart sound signals and electrocardiogram signals to remove background noise;
an external electronic computing device, communicatively coupled to said wearable stethoscope to acquire said pre-processed electrocardiogram signals and said pre-processed body sound signals; and
a cloud database communicatively coupled to said external electronic computing device.

12. The monitoring system of claim 11, wherein said external electronic computing device is a smart phone or a cloud server.

13. The monitoring system of claim 11, wherein said processing unit of said external electronic computing device is configured to execute instructions stored in a storage device of said external electronic computing device to perform screening process of psychogenic diseases, wherein said process includes:
performing data analysis and processing on said pre-processed body sound signals and electrocardiogram signals that are fed in without background noise to obtain corresponding body sound data and electrocardiogram data;
combining heart sound data of said body sound data and electrocardiogram data with user's physiological parameters and medical records for feature extraction, and obtaining related feature vectors;
inputting said related feature vectors into a screening model to obtain an evaluation value, and giving corresponding suggestions based on said evaluation value; and
uploading verification results to a remote data database by said user after screening, expanding said existing training samples to further optimize said parameters of said screening model.

14. The monitoring system of claim 13, wherein verification results include said related feature vectors obtained from said feature extraction, evaluation value and said corresponding suggestions.

15. The monitoring system of claim 13, wherein said screening model includes steps of comparing said relevant physiological parameters between patients and healthy persons.

16. The monitoring system of claim 11, wherein said body sound signals are collected by a sound sensing device of said wearable stethoscope, and said electrocardiogram signals are collected by an electrocardiogram sensing device of said wearable stethoscope.

17. A wearable stethoscope comprising:
an electrocardiographic measuring device to collect electrocardiographic signals of a body;
a processing unit powered by a power source and coupled to said electrocardiographic measuring device to pre-process said electrocardiographic signals to remove background noise; and
a transmission device coupled to said processing unit to transmit said electrocardiographic signals to an external device for personal recognition.

18. The wearable stethoscope of claim 17, wherein said external device is on-board driver recognition system.

19. The wearable stethoscope of claim 17, wherein said power source is coordinated with a power management device to optimize overall power usage.

20. The wearable stethoscope of claim 17, wherein said wireless transmission device is a wireless transceiver connected to an antenna and is configured to transmit and receive data through a wireless telecommunication channel.
